(19) United States
(12) Patent Application Publication
(10) Pub. No.: US 2022/0016099 A1
(43) Pub. Date: Jan. 20, 2022

(54) BENZIMIDAZOLE OR BENZOXAZOLE DERIVATIVES FOR PREVENTING AND TREATING CENTRAL NERVOUS SYSTEM DISEASE, DIABETES, AND COMPLICATIONS THEREOF

(71) Applicant: TRINUERO, Seoul (KR)

(72) Inventors: Younggeun SHIN, Daejeon (KR); Minho PARK, Daejeon (KR); Seokho SHIN, Paju-si (KR); Jinju BYEON, Sejong-si (KR); Jangmi CHOI, Buyeo-gun (KR); Nahye KIM, Cheonan-si (KR); Yuri PARK, Daejeon (KR); Jeonghyeon LIM, Goyang-si (KR); Minjae PARK, Daejeon (KR); Seojin PARK, Daejeon (KR); Byeongill LEE, Daejeon (KR)

(73) Assignee: TRINUERO, Seoul (KR)

(21) Appl. No.: 17/300,000

(22) PCT Filed: Aug. 14, 2019
(86) PCT No.: PCT/KR2019/010384

§ 371 Date: Jun. 4, 2021

Related U.S. Application Data
(60) Provisional application No. 62/776,204, filed on Dec. 6, 2018.

(30) Foreign Application Priority Data
May 31, 2019 (KR) 10-2019-0064588

(51) Int. Cl. A61K 31/4439; A61K 31/4184; A61K 31/423; A61K 31/404

(52) U.S. Cl. CPC A61K 31/4439; A61K 31/404; A61K 31/423; A61K 31/4184

(57) ABSTRACT

The present invention provides a pharmaceutical composition for preventing and treating central nervous system (CNS) diseases, diabetes and complications thereof, the pharmaceutical composition comprising, as an active ingredient, a benzimidazole or benzoxazole derivative having the structure of chemical formula 1 or a pharmaceutically acceptable salt thereof. The benzimidazole or benzoxazole derivative used as an active ingredient in the pharmaceutical composition of the present invention can, via an Nrf2 activation mechanism caused by the Keap1-Nrf2-ARE pathway and via an activation pathway of glutamate transporters such as GLT-1/EAAT and GLAST, inhibit oxidative damage in the central nervous system and the pancreas while inducing production of an antioxidant such as glutathione, thereby maximizing the effect of treating central nervous system diseases, diabetes and complications thereof.

This application claims benefit of priority based on U.S. Provisional Application No. 62/776,204 filed on Dec. 6, 2018, and Korean Patent Application No. 10-2019-0064588 filed on May 31, 2019, the disclosures of which are incorporated herein by reference in their entireties.

### TECHNICAL FIELD

The present invention relates to a pharmaceutical composition for preventing or treating a central nervous system disease, diabetes and complications thereof, comprising a benzimidazole or benzoxazole derivative.

### BACKGROUND ART

The central nervous system (CNS) disease refers to a group of diseases encompassing abnormalities of the nervous system. Depending on the etiology, the CNS disease may be classified into behavioral disorders such as Parkinson's disease, various dementias (neurodegenerative brain diseases) such as Alzheimer's disease, nervous system tumors, demyelinating diseases such as multiple sclerosis, prion diseases, infections of the brain or spinal cord, such as meningitis, schizophrenia, and the like. In particular, the neurodegenerative brain disease is a disease caused by abnormalities of neurons that constitute the brain, spinal cord, or the like, and this is a complex disease in which various factors work. Thus, research on therapeutic mechanism thereof is also conducted in various ways.

Diabetes mellitus is divided mainly into two types, that is, type 2 diabetes mellitus caused by risk factors such as obesity linked to dietary habits, metabolic syndrome, and hypertension, and type 1 diabetes mellitus caused by autoimmune destruction of pancreatic insulin-producing beta cells. For the diabetes mellitus, each type has different etiology and time of onset; however, damage to peripheral organs may occur due to elevated blood sugar levels. It is possible that this disease causes a variety of complications such as diabetic nephropathy, diabetic retinopathy, and diabetic cardiomyopathy. Recently, it has been known that an imbalance in redox homeostasis acts as a pathogenesis mechanism for diabetic diseases and complications thereof. It has been found that diabetic patients have increased intracellular reactive oxygen species (ROS), and thus have increased DNA damage. Based on this finding, research on therapeutic mechanism thereof is actively conducted.

The cause of neurodegenerative diseases and diabetic complications may be associated with ROS-induced oxidative damage to cells or inflammatory apoptosis. In particular, lipids make up 60% or more of the brain, and form, through oxidation by free radicals, reactive oxygen species, such as hydrogen peroxide, superoxide anion, and hydroxyl radical, which are highly reactive, thereby causing apoptosis of brain cells. Such oxidative damage and inflammatory apoptosis in the brain accelerate progression of neurodegenerative central nervous system diseases such as Alzheimer's disease, Parkinson's disease, and dementia.

As a blood sugar level rises, pancreatic beta cells undergo oxidative phosphorylation, which consumes a large amount of oxygen, to secrete insulin. This, in turn, increases risk factors such as oxidative damage and production of reactive oxygen species. In addition, the pancreatic beta cells produce fewer antioxidant enzymes, such as catalase, superoxide dismutase (SOD), and glutathione peroxidase (GPx), than other tissues; and this accelerates progression of diabetic complications due to oxidative damage and inflammatory apoptosis when diabetes develops.

Meanwhile, in the Keap1-Nrf2 protein complex, it is known that Nrf2 (nuclear factor-E2-related factoR2) is a redox-sensitive transcription factor and, when activated, plays an important role in regulating expression of antioxidant-related proteins that have a protective action against oxidative stress, inflammatory responses, and active apoptosis. Under normal conditions, Nrf2 binds with Keap1 (Kelch-like ECH-related protein 1) to form a Keap1-Nrf2 complex where Nrf2 exists in an inactive state. However, when oxidative damage occurs, Nrf2 is separated from Keap1, is activated, and migrates into the nucleus. In the nucleus, Nrf2 can bind to an antioxidant response element (ARE) and induce expression of the target genes that are antioxidant-related genes such as γ-glutamylcysteine ligase (GCL), glutathione-S-transferase (GST), NAD(P)H: quinone oxidoreductase 1 (NQO1), and heme oxygenase-1 (HO-1) (see Kang et al., (2005), Antioxid. Redox. Signal, 7, 1664-1673; Steele et al., (2013), Redox. Biol, 1, 441-445; Chen et al., (2009), Proc. Natl. Acad. Sci. U.S.A 106, 2933-2938; Innamorato et al., (2008), J. Immunol. 181, 680-689; Li et al., (2007), Toxicol Lett, 171, 87-98; Scapagnini et al., (2011), Mol. Neurobiol. 44, 192-201).

In addition, activity of Nrf2 in the brain plays an important role in treating a neurodegenerative central nervous system disease. It has been reported that many patients with a CNS disease have a remarkably low level of glutathione (GSH), which inhibits oxidative damage and inflammation of neurons in the brain, as compared with normal people (see Hybertson et al., (2011), Mol. Aspects. Med, 32, 234-246; Raffa et al., (2011), BMC. Psychiatry, 11, 124; Gawryluk et al., (2011), Int. J. Neuropsychopharmacol. 14, 123-130); and it has been known that intravenous injection or nasal spray of GSH or a precursor thereof improves a CNS disease (see Mischley et al., NPJ. Parkinsons. Dis., 2, 16002; Sechi et al., (1996), Prog. Neuropsychopharmacol. Biol. Psychiatry., 20, 1159-1170; Hauser et al., (2009), Mov. Disord., 24, 979-983). However, pharmacokinetic problems such as low bioavailability and short half-life of GSH make it difficult to administer GSH itself or a precursor thereof.

Glutamic acid transporters cause the concentration of glutamic acid, which is an excitatory neurotransmitter in the brain, to be kept low, and thus play a role in inhibiting excitotoxicity that may occur in the extracellular space. It has been reported that in a case where abnormality occurs in expression of glutamic acid transporters (for example, GLT-1/EAAT and GLAST) related to glutamic acid homeostasis, various CNS diseases develop due to neuronal damage and neurodegeneration in the brain (see Kim et al., (2011), J. Cell. Physiol., 226(10), 2484-2493; Karki et al., (2015), Neurochem. Res., 40(2), 380-388). Recently, it has been known that substances, which induce activation and expression of glutamic acid transporters, promote GSH production in the brain, and thus improve oxidative damage and neurotoxicity caused by methylmercury (MeHg) or the like (see Fontana., (2015), J. Neurochem., 134, 982-1007; Deng et al., (2012), Oxid. Med. Cell. Longev.).

Conventional therapeutic agents for neurodegenerative brain diseases such as Alzheimer's disease and dementia are divided into acetylcholinesterase (AchE) inhibitors that inhibit action of AchE, which is an enzyme that catalyzes breakdown of acetylcholine (neurotransmitter), and N-methyl-D-aspartate (NMDA) receptor antagonists that inhibit the glutamate transport system which causes excitatory toxicity in nerves and inhibits brain plasticity. Drugs approved by the U.S. Food and Drug Administration (FDA) to date include AchE inhibitors such as donepezil (Aricept), rivastigmine (Exelon), galantamine (Reminyl), and tacrine (Cognex), and Memantine (Ebixa) that is an NMDA receptor antagonist.

The conventional therapeutic agents have an effect of improving decreased cognitive function, but the effect is insignificant or temporary. Such therapeutic agents focus on relieving symptoms caused by neuronal damage. For example, therapeutic agents for Alzheimer's disease, in which amyloid accumulates in the brain and ultimately causes massive brain cell necrosis, is primarily aimed at relieving symptoms; and drugs under development also focus on inhibiting beta-amyloid aggregation and brain cell necrosis.

Meanwhile, in the conventional treatment of diabetes, insulin, a substance that lowers blood sugar, is basically used, with additional drugs being used in comprehensive consideration of blood pressure, blood cholesterol level, glycosylated hemoglobin level, fasting blood sugar level, and the like, which are the risk factors for diabetic complications. Currently, the following drugs with various mechanisms have been approved as therapeutic agents for diabetes: metformin (Glucophage) which is an AMP-activated protein kinase (AMPK) activator; exenatide (Byetta) which is a glucagon-like peptide-1 (GLP1) inhibitor; empagliflozin (Jardiance) which is a sodium-glucose co-transporter 2 inhibitor (SGLT2) inhibitor; and the like.

The conventional drugs have an effect of improving a blood sugar level, and treatment with additional drugs is combined therewith to treat accompanying complications. Nevertheless, it is difficult to prevent or delay progression of complications in diabetic patients.

Therefore, there is a need to develop therapeutic agents for neurological diseases and for diabetes and complications thereof, the therapeutic agents being based on various new mechanisms.

### Technical Problem

As a result of intensive studies to develop a therapeutic agent for neurological diseases which is based on a new mechanism, the present inventors have found that certain benzimidazole or benzoxazole derivatives can, via an Nrf2 activation mechanism caused by the Keap1-Nrf2-ARE pathway and via an activation pathway of glutamate transporters such as GLT-1/EAAT and GLAST, inhibit oxidative damage in the central nervous system and the pancreas while inducing production of an antioxidant such as glutathione, thereby maximizing the effect of treating a central nervous system disease, diabetes and complications thereof. Based on this finding, the present inventors have completed the present invention.

Accordingly, an object of the present invention is to provide a pharmaceutical composition for preventing or treating a central nervous system disease, diabetes and complications thereof, comprising a benzimidazole or benzoxazole derivative.

### Solution to Problem

According to an aspect of the present invention, there is provided a pharmaceutical composition for preventing or treating a central nervous system (CNS) disease, diabetes and complications thereof, the pharmaceutical composition comprising, as an active ingredient, a benzimidazole or benzoxazole derivative having the structure of Formula 1, or a pharmaceutically acceptable salt thereof:

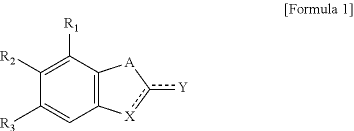

In the formula,

R₁ is hydrogen, a halogen atom, a hydroxyl group, or substituted or unsubstituted C₁-C₆ alkyl,

R₂ and R₃ are each independently hydrogen, a halogen atom, hydroxyl, a C₁-C₆ alkyl group, substituted or unsubstituted C₁-C₆ alkoxy, sulfonyl, sulfonic acid, or nitro, or R₂ and R₃ combine together to form an aryl ring,

A is C₁-C₆ alkylene, NH, or (C₁-C₆ alkyl)-N,
X is O, N, or NH, and
Y is O, S, —SO—(CH₂)ₙ—R₄, —S—(CH₂)ₙ—R₄, —SO₂—(CH₂)ₙ—R₄, or —S—R₅, wherein n is an integer of 1 to 3; R₄ is pyridine unsubstituted or substituted with at least one selected from the group consisting of C₁-C₆ alkyl, C₁-C₆ alkoxy, halo-C₁-C₆ alkoxy, and C₁-C₆ alkoxy-C₁-C₆ alkoxy; and R₅ is hydrogen or C₁-C₆ alkyl.

### Advantageous Effects of Invention

In the pharmaceutical composition for preventing or treating a central nervous system disease, diabetes and complications thereof, of the present invention, the benzimidazole or benzoxazole derivative used as an active ingredient can, via an Nrf2 activation mechanism caused by the Keap1-Nrf2-ARE pathway and via an activation pathway of glutamate transporters such as GLT-1/EAAT and GLAST, inhibit oxidative damage in the central nervous system and the pancreas while inducing production of an antioxidant such as glutathione, thereby maximizing the effect of treating a central nervous system disease, diabetes and complications thereof.

### DETAILED DESCRIPTION OF INVENTION

Hereinafter, the terms or words used in the present specification and claims should not be limitedly interpreted as their usual or dictionary meanings, but should be interpreted as meanings and concepts, which are consistent with the technical spirit of the present invention, based on the principle that inventors can appropriately define concepts of terms to describe their invention in the best way.

The pharmaceutical composition for preventing or treating a central nervous system (CNS) disease, diabetes and complications thereof, according to an embodiment of the present invention, comprises, as an active ingredient, a benzimidazole or benzoxazole derivative having the structure of Formula 1, or a pharmaceutically acceptable salt thereof:

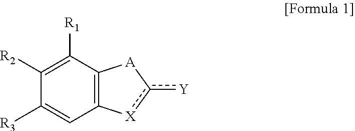

In the formula,

R₁ is hydrogen, a halogen atom, a hydroxyl group, or substituted or unsubstituted C₁-C₆ alkyl,

R₂ and R₃ are each independently hydrogen, a halogen atom, hydroxyl, a C₁-C₆ alkyl group, substituted or unsubstituted C₁-C₆ alkoxy, sulfonyl, sulfonic acid, or nitro, or R₂ and R₃ combine together to form an aryl ring,

A is C₁-C₆ alkylene, NH, or (C₁-C₆ alkyl)-N,

X is O, N, or NH, and
Y is O, S, —SO—(CH₂)ₙ—R₄, —S—(CH₂)ₙ—R₄, —SO₂—(CH₂)ₙ—R₄, or —S—R₅, wherein n is an integer of 1 to 3; R₄ is pyridine unsubstituted or substituted with at least one selected from the group consisting of C₁-C₆ alkyl, C₁-C₆ alkoxy, halo-C₁-C₆ alkoxy, and C₁-C₆ alkoxy-C₁-C₆ alkoxy; and R₅ is hydrogen or C₁-C₆ alkyl.

In an embodiment of the present invention, R₂ and R₃ may each independently be a C₁-C₆ alkyl group substituted with a halogen atom (for example, F, C₁, Br, or I), or C₁-C₆ alkoxy substituted with a halogen atom (for example, F, C₁, Br, or I).

In an embodiment of the present invention, R₂ and R₃ may each independently be a C₁-C₆ alkyl group substituted with F, or C₁-C₆ alkoxy substituted with F.

In another embodiment of the present invention, R₂ and R₃ may each independently be —NO₂, —(NO)OH, or —(SO₂)OH.

In another embodiment of the present invention, R₂ and R₃ may combine together to form a benzene ring or a phenyl ring.

In an embodiment of the present invention, R₄ may be pyridine substituted with at least one selected from the group consisting of C₁-C₆ alkyl and C₁-C₆ alkoxy.

In an embodiment of the present invention, R₄ may be pyridine substituted with at least one selected from the group consisting of C₁-C₆ alkyl and halo-C₁-C₆ alkoxy.

In an embodiment of the present invention, R₄ may be pyridine substituted with at least one C₁-C₆ alkoxy.

In an embodiment of the present invention, R₄ may be pyridine substituted with at least one selected from the group consisting of C₁-C₆ alkyl and C₁-C₆ alkoxy-C₁-C₆ alkoxy.

As used herein, the term “alkyl” refers to a linear or branched saturated hydrocarbon radical chain. Examples thereof include, but are not limited to, methyl, ethyl, n-propyl, isopropyl, n-butyl, isobutyl, t-butyl, n-pentyl, isopentyl, and hexyl.

As used herein, the term “aryl” refers to a benzene ring or a ring system that may be formed by fusion of any one or more substituents.

As used herein, the term “alkoxy” refers to an alkyl group linked to oxygen. Examples thereof include, but are not limited to, methoxy, difluoromethoxy, trifluoromethoxy, ethoxy, n-propoxy, isopropoxy, n-butoxy, and t-butoxy. The alkoxy may be substituted with any substituent, in which the substituent may be selected from the group consisting of C₁-C₃ alkyl optionally having 1 to 3 fluorine substituents, C₂-C₃ alkenyl, C₂-C₃ alkynyl, C₁-C₂ alkoxy optionally having 1 to 3 fluorine substituents, sulfanyl, sulfinyl, sulfonyl, oxo, hydroxy, mercapto, amino, guanidino, carboxy, aminocarbonyl, aryl, aryloxy, heteroaryl, heteroaryloxy, heterocycle, aminosulfonyl, sulfonylamino, carboxyamide, ureido, nitro, cyano, and halogen.

As used herein, the term “alkylene” refers to an alkyl radical in which one hydrogen atom at any position is replaced by one additional binding site to form a divalent moiety.

As used herein, the term “sulfonyl” refers to the group —S(O)₂Rₐ, where Rₐ may be alkyl, hydroxyl, or aryl as defined above.

As used herein, the term “sulfonic acid” refers to the group —SO₂—OH.

As used herein, the term “amino” refers to the group —NH₂, and the amino group may be substituted with alkyl, hydroxyl, or aryl.

As used herein, the term “nitro” refers to the group —NO₂, and the nitro group may be protonated, for example, as shown in

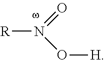

In an embodiment of the present invention, in a case where Y in Formula 1 is O or S,

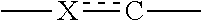

may be a single bond, and

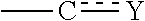

may be a double bond; and
in a case where Y is —SO—(CH₂)—R₄, —S—(CH₂)—R₄, —SO₂—(CH₂)—R₄, or —S—R₅,

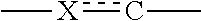

may be a double bond, and

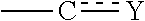

may be a single bond.

In an embodiment of the present invention, the substituent R₄ may be selected from the pyridine structures of Formulas 2a to 2d:

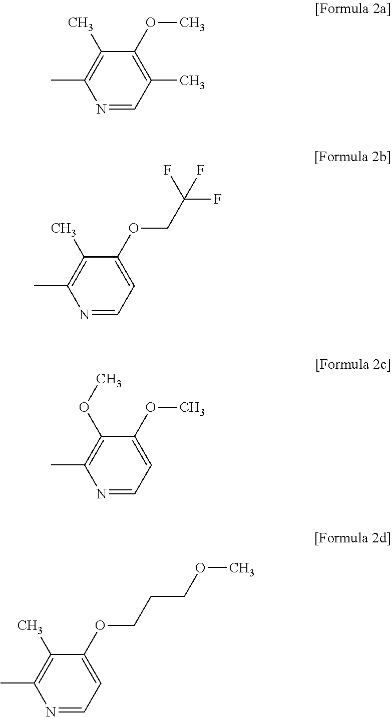

In the formulas, the nitrogen atom of the pyridine ring is unsubstituted or substituted with oxide.

Representative examples of Formula 1 include the following compounds:
1) omeprazole;
2) omeprazole sulfide;
3) omeprazole sulfone;
4) omeprazole N-oxide;
5) 5-methoxy-2-benzimidazolethiol;
6) 5-methoxy-1-methyl-1H-benzimidazole-2-thiol;
7) 5-methoxy-2-(methylthio)-1H-benzimidazole;
8) lansoprazole;
9) lansoprazole sulfide;
10) lansoprazole sulfone;
11) lansoprazole N-oxide;
12) 2-mercaptobenzimidazole;
13) 1-methyl-1H-benzimidazole-2-thiol;
14) pantoprazole;
15) pantoprazole sulfide;
16) pantoprazole sulfone;
17) pantoprazole N-oxide;
18) 5-difluoromethoxy-2-mercaptobenzimidazole;
19) rabeprazole;
20) rabeprazole sulfide;
21) rabeprazole sulfone;
22) rabeprazole N-oxide;
23) chlorzoxazone;
24) 4-methyl-1H-benzimidazole-2-thiol;
25) 2-sulfanyl-1H-benzimidazole-5-sulfonic acid;
26) 5-chloro-1,3-dihydrobenzimidazol-2-one;
27) 5-hydroxyindolin-2-one;
28) 4,5,6-trichloro-3H-a,3-benzoxazol-2-one;
29) 6-methoxybenzo[d]oxazol-2(3H)-one;
30) 2-mercapto-5-nitrobenzimidazole;
31) 6-hydroxychlorzoxazone;
32) 5-chloro-2-mercaptobenzimidazole;
33) 1,3-dihydrobenzo[f]benzimidazole-2-thione; and
34) 5,6-dimethoxy-1H-benzo[d]imidazol-2-thiol.

Specifically, Compounds 1 to 34 are compounds of Formula 1 having substituents as shown in Table 1.

TABLE 1

| Compound | R₁ | R₂ | R₃ | A | X | Y (including R₄ and R₅) |
|---|---|---|---|---|---|---|
| 1 (Omeprazole) | H | H | CH₃—O | NH | N | 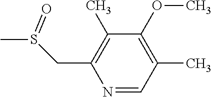 |
| 2 (Omeprazole sulfide) | H | H | CH₃—O | NH | N | 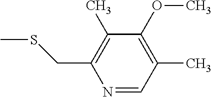 |
| 3 (Omeprazole sulfone) | H | H | CH₃—O | NH | N | 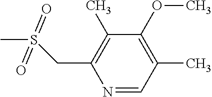 |
| 4 (Omeprazole N- oxide) | H | H | CH₃—O | NH | N | 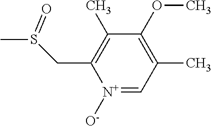 |
| 5 | H | H | CH₃—O | NH | N | —SH |
| (5-Methoxy-2- | | | | | | |
| benzimidazolethiol) | | | | | | |
| 6 | H | H | CH₃—O | N—CH₃ | N | —SH |
| (5-Methoxy-1-methyl- | | | | | | |
| 1H-benzimidazole-2- | | | | | | |
| thiol) | | | | | | |
| 7 | H | H | CH₃—O | NH | N | —S—CH₃ |
| (5-Methoxy-2- | | | | | | |
| (methylthio)-1H- | | | | | | |
| benzimidazole) | | | | | | |
| 8 (Lansoprazole) | H | H | H | NH | N | 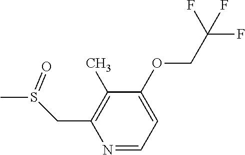 |
| 9 (Lansoprazole sulfide) | H | H | H | NH | N | 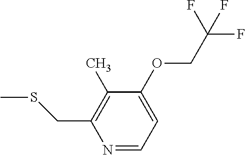 |
| 10 (Lansoprazole sulfone) | H | H | H | NH | N | 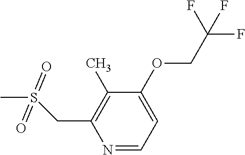 |
| 11 (Lansoprazole N- oxide) | H | H | H | NH | N | 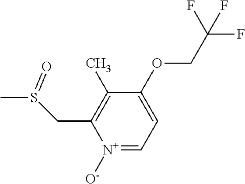 |
| 12 | H | H | H | NH | N | —SH |
| (2- | | | | | | |
| Mercaptobenzimidazole) | | | | | | |
| 13 | H | H | H | N—CH₃ | N | —SH |
| (1-Methyl-1H- | | | | | | |
| benzimidazole-2- | | | | | | |
| thiol) | | | | | | |
| 14 (Pantoprazole) | H | H | CHF₂—O | NH | N | 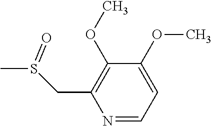 |
| 15 (Pantoprazole sulfide) | H | H | CHF₂—O | NH | N | 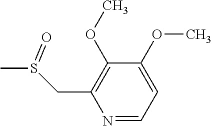 |
| 16 (Pantoprazole sulfone) | H | H | CHF₂—O | NH | N | 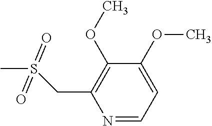 |
| 17 (Pantoprazole N- oxide) | H | H | CHF₂—O | NH | N | 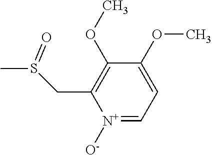 |
| 18 | H | H | CHF₂—O | NH | N | —SH |
| (5-Difluoromethoxy- | | | | | | |
| 2- | | | | | | |
| mercaptobenzimidazole) | | | | | | |
| 19 (Rabeprazole) | H | H | H | NH | N | 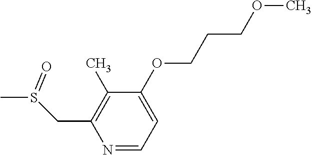 |
| 20 (Rabeprazole sulfide) | H | H | H | NH | N | 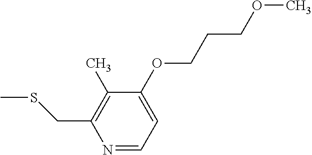 |
| 21 (Rabeprazole sulfone) | H | H | H | NH | N | 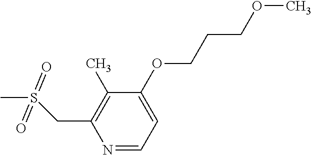 |
| 22 (Rabeprazole N- oxide) | H | H | H | NH | N | 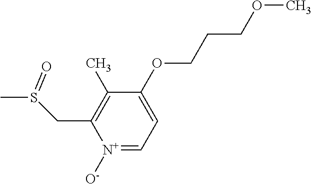 |
| 23 | H | Cl | H | NH | O | ═O |
| (Chlorozoxazone) | | | | | | |
| 24 | CH₃ | H | H | NH | NH | ═S |
| (4-Methyl-1H- | | | | | | |
| benzimidazole-2- | | | | | | |
| thiol) | | | | | | |
| 25 | H | SO₃H | H | NH | NH | ═S |
| (2-Sulfanyl-1H- | | | | | | |
| benzimidazole-5- | | | | | | |
| sulfonic acid) | | | | | | |
| 26 | H | Cl | H | NH | NH | ═O |
| (5-Chloro-1,3- | | | | | | |
| dihydrobenzimidazol- | | | | | | |
| 2-one) | | | | | | |
| 27 | H | HO | H | CH₂ | NH | ═O |
| (5-Hydroxyindolin-2- | | | | | | |
| one) | | | | | | |
| 28 | Cl | Cl | Cl | NH | O | ═O |
| (4,5,6-trichloro-3H- | | | | | | |
| a,3-benzoxazol-2- | | | | | | |
| one) | | | | | | |
| 29 | H | H | CH₃—O | NH | O | ═O |
| (6- | | | | | | |
| Methoxybenzo[d] | | | | | | |
| oxazol-2(3H)-one) | | | | | | |
| 30 | H | OH—NO | H | NH | NH | ═S |
| (2-Mercapto-5- | | | | | | |
| nitrobenzimidazole) | | | | | | |
| 31 | H | Cl | OH | NH | O | ═O |
| (6-Hydroxy- | | | | | | |
| chlorzoxazone) | | | | | | |
| 32 | H | Cl | H | NH | NH | ═S |
| (5-Chloro-2- | | | | | | |
| mercaptobenzimidazole) | | | | | | |
| 33 (1,3- Dihydrobenzo[f] benzimidazole-2-thione) | H | 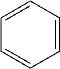 | | NH | H | ═S |
| 34 | H | CH₃—O | CH₃—O | NH | N | —SH |
| (5,6-Dimethoxy-1H- | | | | | | |
| benzo[d]imidazol-2- | | | | | | |
| thiol) | | | | | | |

The benzimidazole or benzoxazole derivative of Formula 1 used as an active ingredient in the pharmaceutical composition according to an embodiment of the present invention can, via an Nrf2 activation mechanism caused by the Keap1-Nrf2-ARE pathway and via an activation pathway of glutamate transporters such as GLT-1/EAAT and GLAST, prevent oxidative damage in the central nervous system.

The benzimidazole or benzoxazole derivative of Formula 1 used as an active ingredient in the pharmaceutical composition of the present invention can, through a simple treatment such as by oral administration, intraperitoneal administration, or intravenous administration, inhibit oxidative damage in the central nervous system while inducing production of an antioxidant such as glutathione so that an increased GSH level is achieved, which may be associated with antioxidant mechanisms caused by activation of the Nrf2 pathway and activation of the glutamate transporter pathway.

Accordingly, the benzimidazole or benzoxazole derivative of Formula 1 can, via an Nrf2 activation mechanism caused by the Keap1-Nrf2-ARE pathway and via an activation pathway of glutamate transporters such as GLT-1/EAAT and GLAST, increase a concentration of GSH in the brain, and thus prevent neuronal damage, thereby maximizing its central nervous system disease therapeutic function. The pharmaceutical composition of the present invention, which comprises the derivative as an active ingredient, can be effectively used for prevention or treatment of various CNS diseases such as depression, anxiety disorder, bipolar disorder, attention deficit hyperactivity disorder (ADHD), autism, stress-related disorders, psychotic disorders such as schizophrenia, neurological diseases such as Parkinson's disease, neurodegenerative diseases such as Alzheimer's disease, epilepsy, migraine, hypertension, impaired or abnormal body temperature homeostasis, disrupted sleep and circadian rhythm, and cardiovascular diseases.

In addition, the benzimidazole or benzoxazole derivative of Formula 1 used as an active ingredient in the pharmaceutical composition of the present invention can, via an Nrf2 activation mechanism caused by the Keap1-Nrf2-ARE pathway and via an activation pathway of glutamate transporters such as GLT-1/EAAT and GLAST, inhibit oxidative damage not only in the central nervous system but also in the pancreas while inducing production of GSH, and additionally increase insulin sensitivity, which makes the derivative effective in treatment of diabetes and complications thereof.

The complication of diabetes includes, but is not limited to, neurological diseases, hyperlipidemia, hypertension, retinopathy, renal failure, and cardiomyopathy.

The present invention includes not only the benzimidazole or benzoxazole derivative of Formula 1, but also pharmaceutically acceptable salts thereof, and all possible solvates, hydrates, racemates, or stereoisomers which may be prepared therefrom.

The benzimidazole or benzoxazole derivative of the present invention may be used in the form of a pharmaceutically acceptable salt. As the salt, an acid addition salt formed by a pharmaceutically acceptable free acid is useful. The acid addition salts are obtained from inorganic acids, such as hydrochloric acid, nitric acid, phosphoric acid, sulfuric acid, hydrobromic acid, hydroiodic acid, nitrous acid, and phosphorous acid, and nontoxic organic acids, such as aliphatic mono- and dicarboxylates, phenyl-substituted alkanoates, hydroxyalkanoates and alkanedioates, aromatic acids, and aliphatic and aromatic sulphonic acids.

Each of the acid addition salts according to the present invention may be prepared using a conventional method such as dissolving the benzimidazole or benzoxazole derivative represented by Formula 1 in an excess acid aqueous solution to form a salt and precipitating the salt using a water-miscible organic solvent, such as methanol, ethanol, acetone, or acetonitrile. In addition, the acid addition salt may be prepared by evaporating a solvent or excess acid in the produced mixture to dryness, or subjecting the precipitated salt to suction filtration.

In addition, pharmaceutically acceptable metal salts may be prepared using bases. An alkali metal or alkali earth metal salt is obtained, for example, by dissolving the compound in an excess alkali metal hydroxide or alkali earth metal hydroxide solution, filtering the undissolved compound salt, and evaporating the filtrate to dryness. Here, as the metal salt, it is pharmaceutically suitable to prepare a sodium, potassium, or calcium salt. In addition, a corresponding silver salt is obtained by reacting an alkali metal or alkaline earth metal salt with a suitable silver salt (for example, silver nitrate).

The pharmaceutical composition according to the present invention may be administered orally (for example, by ingestion or inhalation) or parenterally (for example, by injection, deposition, implantation, or suppository). The injection may be, for example, intravenous, subcutaneous, intramuscular, or intraperitoneal. Depending on the route of administration, the pharmaceutical composition of the present invention may be formulated into tablets, capsules, granules, fine subtilaes, powders, sublingual tablets, suppositories, ointments, injections, emulsions, suspensions, syrups, sprays, or the like. The various types of the pharmaceutical compositions according to the present invention may be prepared by known techniques using pharmaceutically acceptable carriers that are commonly used for respective formulations. Examples of the pharmaceutically acceptable carrier include excipients, binders, disintegrating agents, lubricants, preservatives, antioxidants, isotonic agents, buffers, coating agents, sweetening agents, solubilizing agents, bases, dispersing agents, wetting agents, suspending agents, stabilizers, coloring agents, and the like.

Depending on drug types, the pharmaceutical composition according to the present invention may contain the benzimidazole or benzoxazole derivative of Formula 1, or a pharmaceutically acceptable salt thereof, in 1% to 90% by weight based on a total weight of the composition.

A specific dosage of the pharmaceutical composition of the present invention may vary depending on species of mammals, including humans, to be treated, body weight, sex, severity of disease, physician's judgment, and the like. Preferably, for oral administration, 0.01 to 50 mg of the active ingredient per kg of body weight is administered per day; and for parenteral administration, 0.01 to 10 mg of the active ingredient per kg of body weight is administered per day.

The total daily dosage may be administered once or divided into several times depending on severity of disease, physician's judgment, and the like.

Hereinafter, in order to help understand the present invention, the following examples are provided to describe the present invention in more detail. However, embodiments according to the present invention may be modified into a variety of different forms, and the scope of the present invention should not be construed as being limited to the following examples. The examples of the present invention are provided to those of ordinary skill in the art so that the present invention is described in a more complete manner.

### Example 1: Method for Measuring Nrf2 Activity of Benzimidazole or Benzoxazole Derivatives Using A549-ARE Luciferase Assay

An assay for Nrf2 activity was performed by modification of the method reported in Ilse M. Beck et al., (2013), European Journal of Pharmacology 715. 1-9.

Specifically, A549-ARE cell line was dispensed into a 96-well plate, and then subjected to treatment with a solution obtained by dissolving 10 μM of each compound in Table 2 in dimethyl sulfoxide (DMSO). On the other hand, Compounds 1 and 13 as shown in Table 2 were obtained from Sigma-Aldrich (US); Compounds 2, 5, 8, 9 12, 15, 18 to 20, 23, 25, 30, and 32 were obtained from Tokyo Chemical Industry Co., Ltd. (JP); Compound 3 was obtained from Labseeker (CN); Compound 4 was obtained from BOC Science (US); Compounds 6 and 7 were obtained from Cambridge (US); Compounds 10, 11, 16, 17, 21, and 22 were obtained from Toronto Research Chemicals (CA); Compound 14 was obtained from Selleckchem (US); Compounds 24, 26, 27, and 31 were obtained from BLD Pharm (CN); Compounds 28 and 33 were obtained from Mcule (US); Compound 29 was obtained from Chemshuttle (US); and Compound 34 was obtained from Alinda (RU).

Thereafter, the 96-well plate was treated with 30 μl of luciferase lysis buffer (90.8 mM K₂HPO₄, 9.2 mM KH₂PO₄, 0.1% Triton X-100), and then incubated for 30 minutes or longer so that cell lysis proceeded. The incubated cell lysate was transferred to another 96-well plate (white plate), and then each well was treated with 100 μl of luciferase buffer mixture (20 mM Tricine, 1.07 mM [(MgCO₃)₄Mg(OH)₂.5H₂O], 2.67 mM [MgSO₄.7H₂O], 0.1 mM EDTA, 33.3 mM DTT, 270 μM coenzyme A [CoA], 500 μM D-luciferin, 530 μM ATP). Immediately after the treatment, ELISA analysis was performed to check Nrf2 activity. The results are shown in Table 2.

TABLE 2

| Compound | Chemical structure | Nrf2 activity |
|---|---|---|
| 1 (Omeprazole) | 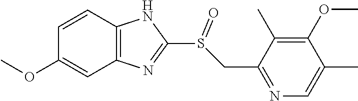 | + |
| 2 (Omeprazole sulfide) | 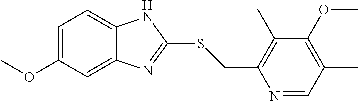 | + |
| 3 (Omeprazole sulfone) | 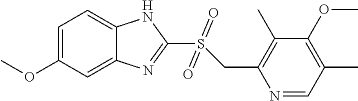 | + |
| 4 (Omeprazole N-oxide) | 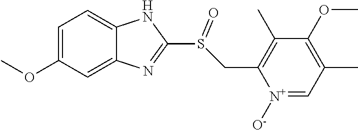 | + |
| 5 (5-Methoxy-2- benzimidazolethiol) | 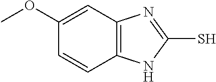 | ++ |
| 6 (5-Methoxy-1-methyl-1H- benzimidazole-2-thiol) | 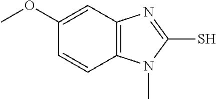 | ++ |
| 7 (5-Methoxy-2-(methylthio)-1H- benzimidazole) | 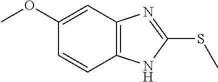 | + |
| 8 (Lansoprazole) | 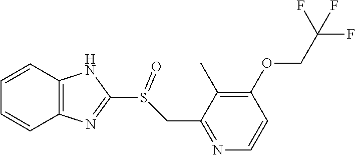 [SMILES: Cc1c(OCC(F)(F)F)ccnc1CS(=O)c1nc2ccccc2[nH]1] | ++++ |
| 9 (Lansoprazole sulfide) | 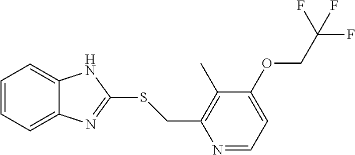 [SMILES: Cc1c(OCC(F)(F)F)ccnc1CSc1nc2ccccc2[nH]1] | +++ |
| 10 (Lansoprazole sulfone) | 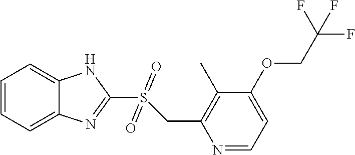 [SMILES: Cc1c(OCC(F)(F)F)ccnc1CS(=O)(=O)c1nc2ccccc2[nH]1] | + |
| 11 (Lansoprazole N-oxide) | 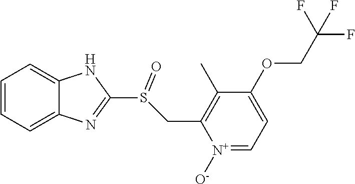 [SMILES: Cc1c(OCC(F)(F)F)cc[n+]([O-])c1CS(=O)c1nc2ccccc2[nH]1] | +++ |
| 12 (2-Mercaptobenzimidazole) | 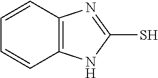 | + |
| 13 (1-Methyl-1H-benzimidazole-2- thiol) | 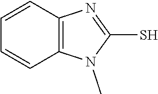 | + |
| 14 (Pantoprazole) | 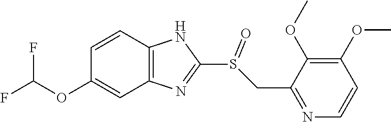 [SMILES: COc1ccnc(CS(=O)c2nc3cc(OC(F)F)ccc3[nH]2)c1OC] | +++ |
| 15 (Pantoprazole sulfide) | 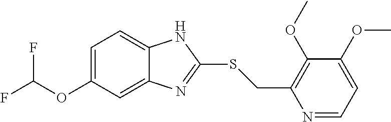 [SMILES: COc1ccnc(CSc2nc3cc(OC(F)F)ccc3[nH]2)c1OC] | + |
| 16 (Pantoprazole sulfone) | 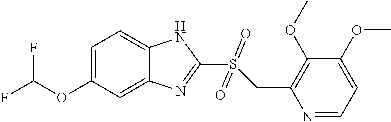 [SMILES: COc1ccnc(CS(=O)(=O)c2nc3cc(OC(F)F)ccc3[nH]2)c1OC] | + |
| 17 (Pantoprazole N-oxide) | 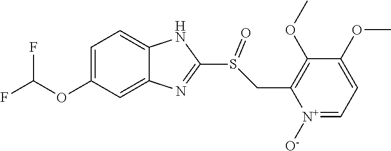 [SMILES: COc1cc[n+]([O-])c(CS(=O)c2nc3cc(OC(F)F)ccc3[nH]2)c1OC] | +++ |
| 18 (5-Difluoromethoxy-2- mercaptobenzimidazole) | 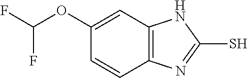 [SMILES: FC(F)Oc1ccc2nc(S)[nH]c2c1] | + |
| 19 (Rabeprazole) | 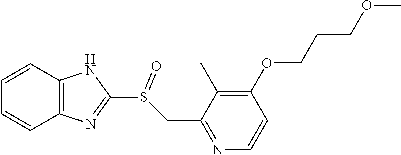 [SMILES: COCCCOc1ccnc(CS(=O)c2nc3ccccc3[nH]2)c1C] | ++++ |
| 20 (Rabeprazole sulfide) | 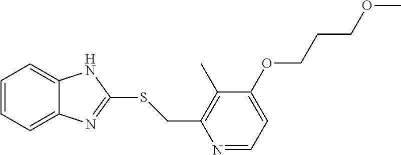 [SMILES: COCCCOc1ccnc(CSc2nc3ccccc3[nH]2)c1C] | ++++ |
| 21 (Rabeprazole sulfone) | 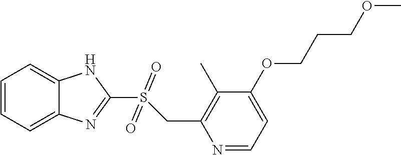 [SMILES: COCCCOc1ccnc(CS(=O)(=O)c2nc3ccccc3[nH]2)c1C] | + |
| 22 (Rabeprazole N-oxide) | 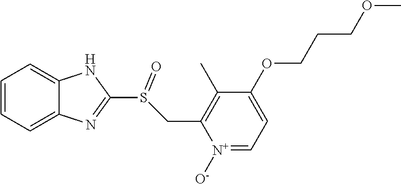 [SMILES: COCCCOc1cc[n+]([O-])c(CS(=O)c2nc3ccccc3[nH]2)c1C] | +++ |
| 23 (Chlorozoxazone) | 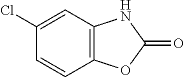 [SMILES: O=c1[nH]c2cc(Cl)ccc2o1] | ++ |
| 24 (4-Methyl-1H-benzimidazole-2- thiol) | 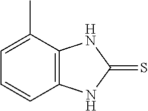 | + |
| 25 (2-Sulfanyl-1H-benzimidazole- 5-sulfonic acid) | 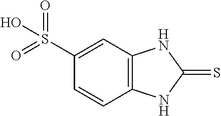 [SMILES: O=S(=O)(O)c1ccc2[nH]c(=S)[nH]c2c1] | + |
| 26 (5-Chloro-1,3- dihydrobenzimidazol-2-one) | 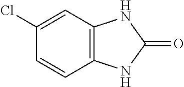 [SMILES: O=c1[nH]c2ccc(Cl)cc2[nH]1] | + |
| 27 (5-Hydroxyindolin-2-one) | 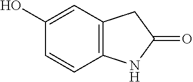 [SMILES: O=C1Cc2cc(O)ccc2N1] | + |
| 28 (4,5,6-trichloro-3H-a,3- benzoxazol-2-one) | 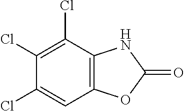 [SMILES: O=c1[nH]c2c(Cl)c(Cl)c(Cl)cc2o1] | + |
| 29 (6-Methoxybenzo[d]oxazol- 2(3H)-one) | 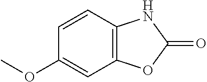 | ++ |
| 30 (2-Mercapto-5- nitrobenzimidazole | 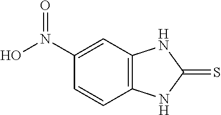 | + |
| 31 (6-Hydroxychlorzoxazone) | 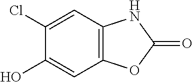 [SMILES: O=c1[nH]c2cc(Cl)c(O)cc2o1] | + |
| 32 (5-Chloro-2- mercaptobenzimidazole) | 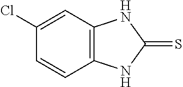 [SMILES: S=c1[nH]c2ccc(Cl)cc2[nH]1] | +++ |
| 33 (1,3- Dihydrobenzo[f]benzimidazole- 2-thione) | 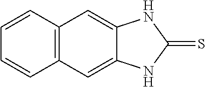 [SMILES: S=c1[nH]c2cc3ccccc3cc2[nH]1] | + |
| 34 (5,6-Dimethoxy-1H- benzo[d]imidazol-2-thiol) | 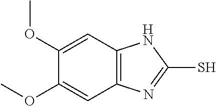 | + |

<Nrf2 activity (%) of compound relative to DMSO using luciferase assay>
0-30% +
30~70% ++
70~100% +++
>100% ++++

As can be seen from Table 2, Compounds 1 to 34 of Formula 1 exhibited Nrf2 activity in the Keap1-Nrf2-ARE pathway as measured by the luciferase assay, indicating that the benzimidazole or benzoxazole derivatives can, via an Nrf2 activation mechanism caused by the Keap1-Nrf2-ARE pathway, inhibit oxidative damage in the central nervous system and the pancreas while inducing production of an antioxidant such as glutathione, which allows such benzimidazole or benzoxazole derivatives to be effectively used for prevention or treatment of a central nervous system disease, diabetes and complications thereof.

1. A pharmaceutical composition for preventing or treating a central nervous system (CNS) disease, diabetes and complications thereof, the pharmaceutical composition comprising, as an active ingredient, a benzimidazole or benzoxazole derivative having the structure of Formula 1, or a pharmaceutically acceptable salt thereof: 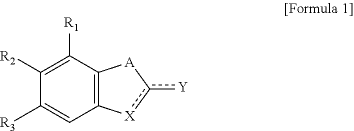
in the formula,
R₁ is hydrogen, a halogen atom, a hydroxyl group, or substituted or unsubstituted C₁-C₆ alkyl,
R₂ and R₃ are each independently hydrogen, a halogen atom, hydroxyl, a C₁-C₆ alkyl group, substituted or unsubstituted C₁-C₆ alkoxy, sulfonyl, sulfonic acid, or nitro, or R₂ and R₃ combine together to form an aryl ring,
A is C₁-C₆ alkylene, NH, or (C₁-C₆ alkyl)-N,
X is O, N, or NH, and
Y is O, S, —SO—(CH₂)ₙ—R₄, —S—(CH₂)ₙ—R₄, —SO₂—(CH₂)ₙ—R₄, or —S—R₅, wherein n is an integer of 1 to 3; R₄ is pyridine unsubstituted or substituted with at least one selected from the group consisting of C₁-C₆ alkyl, C₁-C₆ alkoxy, halo-C₁-C₆ alkoxy, and C₁-C₆ alkoxy-C₁-C₆ alkoxy; and
R₅ is hydrogen or C₁-C₆ alkyl.

2. The pharmaceutical composition according to claim 1, wherein R₄ is selected from the pyridine structures of Formulas 2a to 2d: 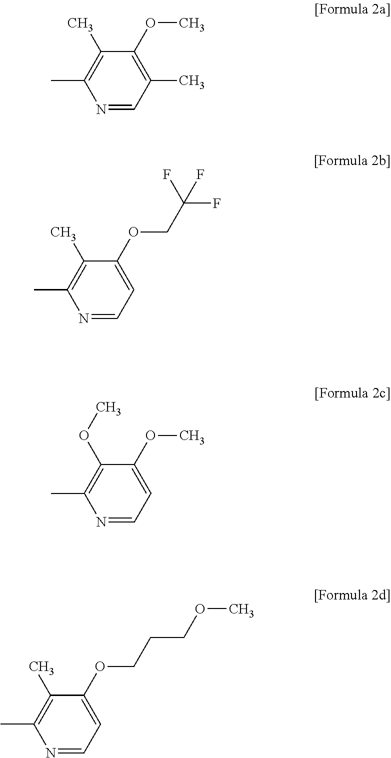
in the formulas, the nitrogen atom of the pyridine ring is unsubstituted or substituted with oxide.

3. The pharmaceutical composition according to claim 1, wherein the benzimidazole or benzoxazole derivative having the structure of Formula 1 includes the following compounds:
1) omeprazole;
2) omeprazole sulfide;
3) omeprazole sulfone;
4) omeprazole N-oxide;
5) 5-methoxy-2-benzimidazolethiol;
6) 5-methoxy-1-methyl-1H-benzimidazole-2-thiol;
7) 5-methoxy-2-(methylthio)-1H-benzimidazole;
8) lansoprazole;
9) lansoprazole sulfide;
10) lansoprazole sulfone;
11) lansoprazole N-oxide;
12) 2-mercaptobenzimidazole;
13) 1-methyl-1H-benzimidazole-2-thiol;
14) pantoprazole;
15) pantoprazole sulfide;
16) pantoprazole sulfone;
17) pantoprazole N-oxide;
18) 5-difluoromethoxy-2-mercaptobenzimidazole;
19) rabeprazole;
20) rabeprazole sulfide;
21) rabeprazole sulfone;
22) rabeprazole N-oxide;
23) chlorzoxazone;
24) 4-methyl-1H-benzimidazole-2-thiol;
25) 2-sulfanyl-1H-benzimidazole-5-sulfonic acid;
26) 5-chloro-1,3-dihydrobenzimidazol-2-one;
27) 5-hydroxyindolin-2-one;
28) 4,5,6-trichloro-3H-a,3-benzoxazol-2-one;
29) 6-methoxybenzo[d]oxazol-2(3H)-one;
30) 2-mercapto-5-nitrobenzimidazole;
31) 6-hydroxychlorzoxazone;
32) 5-chloro-2-mercaptobenzimidazole;
33) 1,3-dihydrobenzo[f]benzimidazole-2-thione; and
34) 5,6-dimethoxy-1H-benzo[d]imidazol-2-thiol.

4. The pharmaceutical composition according to claim 1, wherein the CNS disease is at least one selected from the group consisting of Alzheimer's disease, Parkinson's disease, dementia, schizophrenia, panic disorder, depression, anxiety disorder, bipolar disorder, attention deficit hyperactivity disorder (ADHD), autism, stress-related disorders, psychotic disorders, neurological diseases, neurodegenerative diseases, epilepsy, migraine, hypertension, impaired or abnormal body temperature homeostasis, and disrupted sleep and circadian rhythm.

5. The pharmaceutical composition according to claim 1, wherein the complication of diabetes is at least one selected from the group consisting of hyperlipidemia, hypertension, retinopathy, renal failure, and cardiomyopathy.

6. A method of preventing or treating a central nervous system (CNS) disease, diabetes and complications thereof, comprising
administering the pharmaceutical composition of claim 1 to a subject in need thereof.
